(19) United States
(12) Patent Application Publication
(10) Pub. No.: US 2020/0032435 A1
(43) Pub. Date: Jan. 30, 2020

(54) Warp-Knitted Fabric And Elastic Article

(71) Applicant: 3M INNOVATIVE PROPERTIES COMPANY, St. Paul, MN (US)

(72) Inventor: Kenichiro Morishita, Tokyo (JP)

(21) Appl. No.: 16/484,308

(22) PCT Filed: Feb. 15, 2018
(86) PCT No.: PCT/IB2018/050934

§ 371 Date: Aug. 7, 2019

(30) Foreign Application Priority Data
Feb. 17, 2017 (JP) 2017-028177

(51) Int. Cl. D04B 21/18; A41B 17/00; A41D 13/00; A61F 13/49; A47G 9/02

(52) U.S. Cl. CPC D04B 21/18; A41B 17/00; A61F 2013/00119; A61F 13/49011; A47G 9/02; A41D 13/0015

(57) ABSTRACT

Provided is a warp-knitted fabric configured by a first yarn, a second yarn, and a third yarn, wherein the first yarn and the second yarn are elastic yarns; the third yarn is a nonelastic yarn; and an amplitude A of the first yarn, an amplitude B of the second yarn, and an amplitude C of the third yarn satisfy relationships of A≤B and A≤C. Provided is an elastic article including the warp-knitted fabric above.

### FIELD OF THE INVENTION

The present invention relates to a warp-knitted fabric and an elastic article including such.

### BACKGROUND

Conventionally, as a knitted fabric having favorable elasticity, a warp-knitted fabric such as a double-guide-bar warp-knitted fabric is used. For example, a warp-knitted fabric using a nonelastic yarn as a front yarn and an elastic yarn as a back yarn has favorable elasticity. However, generally, because a double-guide-bar knitted fabric curls readily, thermosetting by a heat-moisture treatment is necessary at a time of manufacturing. This treatment increases manufacturing costs significantly.

As a stretchable fabric, various stacked bodies are also conventionally proposed.

Patent literature 1 describes a breathable waterproof fabric that is formed by stacking at least one layer of a fiber structure consisting of microfibers of an average fiber diameter of 0.01 to 1 μm on a substrate, has a water resistance of no less than 50 kPa, has a moisture permeability of no less than 8,000 g/m2·24 hr, and has an air permeability of no less than 0.2 cc/m2·s.

Patent literature 2 describes a knitted fabric with an excellent cool touch consisting of conjugated fibers, wherein the conjugated fibers include an elastomer resin (A) that has elasticity and an elastomer resin (B) that has elasticity, a tension set of 25 to 70%, and a tensile elongation of 100 to 800%; the elastomer resin (A) is included in a core portion and the elastomer resin (B) is included in a sheath portion; an area ratio between the core portion and the sheath portion at a fiber cross section is 95:5 to 40:60; and a cover factor (CF) of the knitted fabric is 30 to 80% and a qmax value of the knitted fabric is no less than 0.2 J/sec/cm2.

Patent literature 3 describes an elastic web (10) formed including a first pleatable base layer (12) and a multi-strand elastic yarn (14) fixed to the first pleatable base layer by a large number of first fixing areas (16), wherein a portion of the first pleatable base layer (12) is passed between strands (18) of the multi-strand elastic yarn by the first fixing areas.

Patent literature 4 describes a stretch nonwoven fabric, wherein at least one elastic yarn penetrates a nonwoven fabric, is stitched in a weft direction, and forms a weft stitch row.

Patent literature 5 describes a manufacturing method of a composite elastic member of introducing a plurality of elastic members in an elongated state between two layers of a sheet material and, at an area where no elastic member is disposed, forming joining portions that join the two layers of the sheet material by intermittently arranging these joining portions in an elastic direction of the elastic members and a direction orthogonal thereto in relation to a surface of the sheet material.

Patent literature 6 describes an elastic composite sheet configured from an essentially nonelastic sheet member that stretches in a longitudinal direction and a plurality of elastic members that is extended in the longitudinal direction and is lined up separated by a predetermined dimension in a width direction intersecting the longitudinal direction, the elastic members being installed to the sheet member in a state of elongation in the longitudinal direction, wherein the composite sheet has a large number of walls formed by the sheet member and the elastic members repeating undulation alternatingly in the longitudinal direction and the walls extend essentially continuously in the width direction and are lined up at substantially equal intervals in the longitudinal direction.

Patent literature 7 describes a stacked body having an elastic film and a nonwoven fabric ultrasonically joined to each surface of the film, wherein the stacked body has a flat joining region of no greater than about 2%.

Patent literature 8 describes a method of manufacturing an elastically elongatable laminate (19) formed provided with at least three layers (1, 6, 18), provided with the steps of

a) manufacturing a first laminate (8) formed provided with a first nonelastic-fiber nonwoven web (1) and an elastic film (6),

b) activating the first laminate (8) by progressive elongation in at least one direction to make the first laminate (8) elastically elongatable,

c) elongating the activated first laminate (8) in at least one direction by 10 to 200%, and

d) stacking the elongated first laminate (8) on a second nonelastic nonwoven web (18)

Patent literature 9 describes a manufacturing method of a stretchable web stacked body, including the steps of providing a multilayer film including two skin layers and an elastomer core layer sandwiched therebetween, the multilayer film being activated in a machine width direction;

extending the multilayer film in a machine direction past a deformation limit of the skin layers;

stacking a web layer on the multilayer film in a state where the multilayer film is extended in the machine direction; and

forming the stretchable web stacked body by restoring the multilayer film Citation List

### PATENT LITERATURE

[Patent Literature 1] JP 2008-213391 A
[Patent Literature 2] JP 2008-231616 A
[Patent Literature 3] JP 2005-514244 A
[Patent Literature 4] JP H8-81866 A
[Patent Literature 5] JP 2010-260323 A
[Patent Literature 6] JP 2002-371456 A
[Patent Literature 7] JP 2010-195044 A
[Patent Literature 8] JP 2009-536888 A
[Patent Literature 9] JP 2015-529581 A

### SUMMARY

In recent years, both breathability and favorable elasticity are sought of an elastic fabric for use in an elastic article with a usage as a sanitary product, a medical product, a sporting product, or the like. Moreover, with a specific usage such as a waist portion of a diaper of a wearable type, sought is to manufacture an elastic fabric having anisotropic and favorable elasticity (for example, significantly greater elasticity in an MD (machine direction) than a CD (cross direction)), favorable mechanical strength, and favorable breathability at an appropriate cost.

However, in a stacked body formed by, for example, strands of an elastic yarn being formed on a nonwoven fabric, that elasticity is small due to the nonwoven fabric having no elasticity and that, due to the nonwoven fabric adhering to the elastic yarn when the elastic yarn is in a stretched state, the nonwoven fabric is wrinkled in the obtained stacked body are problems. Wrinkles are undesirable in terms of appearance and leaving a mark on the body of a user. Meanwhile, a stacked body of, for example, an elastic film and a nonwoven fabric, while advantageous in terms of being able to be formed as an elastic stacked body of a greater area, has problems where the film has unfavorable breathability and a stacked body of a film and a nonwoven fabric has high manufacturing costs. Moreover, with these stacked bodies, that imparting anisotropy to elasticity is difficult is also a problem. As above, the current situation is that no elastic fabric that can simultaneously satisfy anisotropic and favorable elasticity, favorable mechanical strength, and favorable breathability and can be manufactured at an appropriate cost is obtained.

The present disclosure solves the problems above and has as an object to provide a warp-knitted fabric as an elastic fabric that has anisotropic and favorable elasticity, favorable mechanical strength, and favorable breathability and can be manufactured at an appropriate cost and an elastic article including such.

### Solution to Problem

One aspect of the present invention provides

a warp-knitted fabric configured by a first yarn, a second yarn, and a third yarn, wherein

the first yarn and the second yarn are elastic yarns,

the third yarn is a nonelastic yarn, and

an amplitude A of the first yarn, an amplitude B of the second yarn, and an amplitude C of the third yarn satisfy relationships of A≤B and A≤C.

Another aspect of the present invention provides

an elastic article including the warp-knitted fabric according to one aspect of the present invention, wherein the elastic article is selected from a group consisting of a sanitary product, a medical product, a sports product, underwear, and bedding.

### Advantageous Effects of Invention

According to the means above, providable is, for example, a warp-knitted fabric as an elastic fabric that has anisotropic and favorable elasticity, favorable mechanical strength, and favorable breathability and can be manufactured at an appropriate cost and an elastic article including such.

### BRIEF DESCRIPTION OF DRAWINGS

FIG. 1 A diagram illustrating an example of a knit structure of a warp-knitted fabric of the present disclosure.

FIG. 2 A diagram illustrating the example of the knit structure of the warp-knitted fabric of the present disclosure.

### DESCRIPTION OF EMBODIMENTS

Typical aspects of the present invention are described below, but the present invention is not limited to these aspects. Note that unless indicated otherwise, characteristic values indicated in the present disclosure are values measured according to methods described under [Examples] in the present disclosure or methods understood by a person skilled in the art to be equivalent thereto.

In one aspect, a warp-knitted fabric of the present disclosure is configured by a first yarn, a second yarn, and a third yarn. In such a triple-guide-bar warp-knitted fabric, a problem of curling that arises in, for example, a double-guide-bar warp-knitted fabric is less likely to arise; therefore, a flattening treatment at a time of manufacturing such as thermosetting is unnecessary and an advantage is had in terms of manufacturing costs. In one aspect, the warp-knitted fabric of the present disclosure can be used independently (that is, without being stacked with another member such as an elastic film or a nonwoven fabric) and is advantageous in terms of favorable breathability.

In one aspect, the first yarn and the second yarn are elastic yarns; the third yarn is a nonelastic yarn; and an amplitude A of the first yarn, an amplitude B of the second yarn, and an amplitude C of the third yarn satisfy relationships of A≤B and A≤C.

A form where the first yarn and the second yarn have substantially identical amplitudes as in a situation where both are, for example, 0-0/N-N is anticipated to have large elasticity in a warp direction and small elasticity in a weft direction. Moreover, a combination of an elastic yarn with a relatively small amplitude (first yarn), an elastic yarn with a relatively large amplitude (second yarn), and a nonelastic yarn with a relatively large amplitude (third yarn) is advantageous in terms of obtaining a warp-knitted fabric where elasticity in the warp direction is large, elasticity in the weft direction is small, and curling is suppressed. The second yarn contributes to favorable elasticity, and the third yarn contributes to favorable mechanical strength. Moreover, the first yarn, in the warp direction, contributes to large elasticity by being an elastic yarn and, in one weft direction, contributes to small elasticity by having a small amplitude. That the first yarn and the second yarn are present as elastic yarns is advantageous in terms of both ensuring elasticity and preventing curling. While not intended to be limited to this theory, the following is conceivable as a reason for curling being prevented favorably by the presence of the first yarn and the second yarn. In a situation where a warp-knitted fabric is, unlike that of the present disclosure, configured only by, for example, the first yarn that is an elastic yarn and the third yarn that is a nonelastic yarn, curling is more likely to arise due to the elasticity of the first yarn. Because an elastic yarn is elastic mainly in a travel direction of the yarn, it is thought that curling arises because large elasticity is had in a warp direction due to the first yarn and this elasticity in the warp direction works in a thickness direction. Meanwhile, with the warp-knitted fabric of the present disclosure, because the first yarn and the second yarn are present, elasticity of a certain extent is had in both the warp direction and the weft direction. Because the second yarn, which has a comparatively large amplitude, is disposed in a vicinity of the first yarn, which has a small amplitude (that is, a small spread in the weft direction), it is thought that when the second yarn contracts in the weft direction, it enters a state of making the vicinity of the first yarn somewhat thick along the warp direction such that an action is had where the warp-knitted fabric is supported so the warp-knitted fabric suppresses curling due to contraction in the warp direction. Moreover, because the amplitude of the third yarn and the amplitude of the second yarn are comparatively close, it is thought that by suppressing the second yarn from contracting excessively in the weft direction, an action is had where the warp-knitted fabric is supported so the warp-knitted fabric suppresses curling due to contraction in the warp direction.

In one aspect, the first yarn is a back yarn, the second yarn is a middle yarn, and the third yarn is a front yarn. By this, the third yarn that is a nonelastic yarn can be exposed on a surface of the knitted fabric to impart the surface of the knitted fabric with a smooth and a favorable feel.

In a preferred aspect, the amplitude A of the first yarn is no greater than 2. In a preferred aspect, the first yarn is configured in a knit structure (knit pattern) of a chain stitch. According to these aspects, particularly favorable curling prevention and mechanical strength are imparted to the warp-knitted fabric. In a preferred aspect, the knit structure of the first yarn is an open-loop chain stitch, which contributes to favorable flatness, favorable elasticity in the warp direction, and favorable breathability of the warp-knitted fabric.

In a preferred aspect, in terms of obtaining favorable mechanical strength and small elasticity in the weft direction, the amplitude B of the second yarn and the amplitude C of the third yarn are no greater than 5.

In a preferred aspect, the second yarn is an inserted yarn. By this, particularly favorable elasticity and breathability are imparted to the warp-knitted fabric.

In a preferred aspect, a knit structure of the third yarn has a closed-loop portion. This closed-loop portion contributes to favorable mechanical strength and small elasticity in the weft direction of the warp-knitted fabric. In a preferred aspect, a knit structure of the third yarn consists of only closed loops. In a preferred aspect, the knit structure of the third yarn has no chain-stitched portion; this contributes to favorable breathability and favorable elasticity in the warp direction of the warp-knitted fabric.

In a preferred aspect, at least a portion of the first yarn is configured according to a knit structure of 0-1/1-0. In a preferred aspect, the first yarn may include, as a knit structure including 0-1/1-0, for example, 0-1/1-0/1-0/0-1 or chain stitching of not only open loops but also of closed loops. Among these, 0-1/1-0 contributes to sufficient elasticity in the warp direction.

In a preferred aspect, the second yarn is configured according to a knit structure of 0-0/N-N, 0-1/N-N, N-N/1-0, or N-N/0-0 and the third yarn is configured according to a knit structure of 1-0/(M−1)-M. In one aspect, with the second yarn and the third yarn, 1≤N≤5 (N being an integer) and 1≤M≤5 (M being an integer), and in a preferred aspect, N and M are no less than 2, no less than 3, or no less than 4. N and M being equal or close values—that is, the amplitudes of the second yarn and the third yarn being substantially identical—is particularly advantageous in terms of favorable curling prevention. In a preferred aspect, N and M are equal.

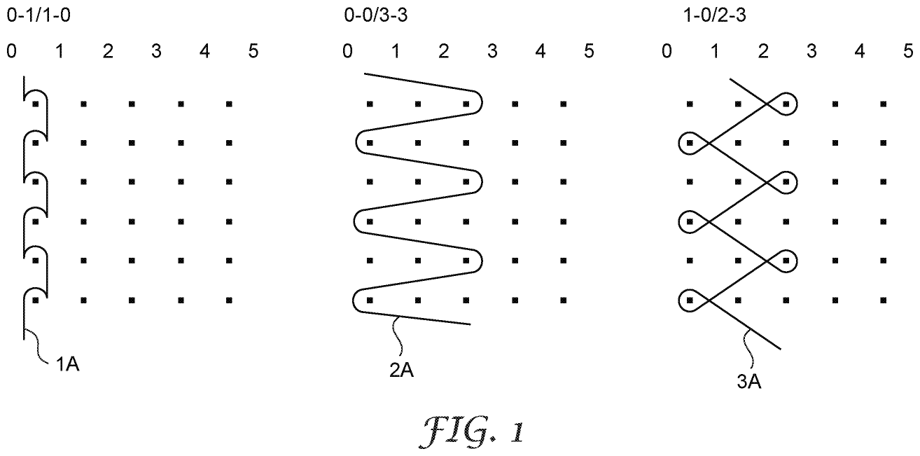

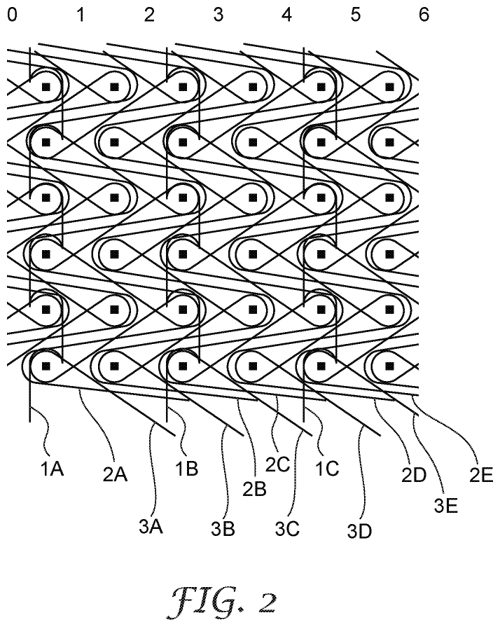

FIGS. 1 and 2 are diagrams illustrating a knit structure of the warp-knitted fabric of the present disclosure. In FIGS. 1 and 2, a situation is illustrated where first yarns 1A, 1B, and 1C have a knit structure of 0-1/1-0; second yarns 2A, 2B, 2C, 2D, and 2E have a knit structure of 0-0/3-3; and third yarns 3A, 3B, 3C, 3D, and 3E have a knit structure of 1-0/2-3; FIG. 1 illustrates the knit structures of the first to third yarns individually, and FIG. 2 illustrates the knit structures of the first to third yarns superimposed. Note that compared to a situation where the second yarn is 0-1/3-3, 3-3/1-0, or 3-3/0-0, it is anticipated that 0-0/3-3 is advantageous in terms of curling prevention; however, in terms of a feel of the warp-knitted fabric, it is anticipated that 0-1/3-3, 3-3/1-0, and 3-3/0-0 are favorable.

In a preferred aspect, the first yarn, the second yarn, and the third yarn are each threaded in a yarn arrangement of complete threading or 1 in, n out (n being an integer). FIG. 2 illustrates a situation where the first yarns 1A, 1B, and 1C are 1 in, 1 out and the second yarns 2A, 2B, 2C, 2D, and 2E and the third yarns 3A, 3B, 3C, 3D, and 3E are each completely threaded. In the illustrated aspect, the yarn arrangements of the first yarn and the second yarn are independently made to be 1 in, n out (n being an integer). A value of n of when the yarn arrangement is made to be 1 in, n out (n being an integer) in the first yarn and the second yarn being smaller is advantageous in terms of mechanical strength of the warp-knitted fabric, but this value being larger is advantageous in terms of elasticity and breathability. The value of n above can be designed independently for the first yarn and the second yarn according to desired characteristics of the warp-knitted fabric. In a preferred aspect, in the first yarn and the second yarn, n above can be made to be in a range of 1 to 16 and 1 to 8 respectively and independently. In a preferred aspect, the yarn arrangement of the third yarn is made to be complete threading for favorable mechanical strength and flatness of the warp-knitted fabric.

In a preferred aspect, the first yarn and the second yarn each include no less than one type selected from a group consisting of a polyurethane and a polyether ester. In a preferred aspect, the first yarn and the second yarn each include a polyurethane or consist of a polyurethane.

In a preferred aspect, the third yarn is a spun yarn or a filament yarn including no less than one type selected from a group consisting of a polyester, a polyolefin, a polyamide, an acrylic, cellulose (for example, regenerated cellulose such as rayon or natural cellulose such as cotton), wool, and linen. In a preferred aspect, the third yarn includes a polyester or consists of a polyester.

In a preferred aspect, to obtain a favorable performance as an elastic article (for example, a fit on the body of a user), a tensile stress of when the warp-knitted fabric is elongated in the warp direction at an elongation rate of 100% is no less than about 0.4 N/25 mm or no less than about 0.5 N/25 mm and, to obtain a favorable performance as an elastic article (for example, ease of wearing), no greater than about 6 N/25 mm or no greater than about 5 N/25 mm.

In a preferred aspect, to obtain a favorable performance as an elastic article (for example, a fit on the body of a user), a tensile stress of when the warp-knitted fabric that is elongated at the elongation rate of 100% in the warp direction is returned to an elongation rate of 50% is no less than about 0.1 N/25 mm or no less than about 0.2 N/25 mm and, to obtain a favorable performance as an elastic article (for example, a favorable wearing feel), no greater than about 4 N/25 mm or no greater than about 35 N/25 mm.

In a preferred aspect, for favorable breathability, an air permeability of the warp-knitted fabric is no less than about 10 cm3/cm2/s or no less than about 50 cm3/cm2/s and, for also obtaining favorable mechanical strength of the warp-knitted fabric, no greater than about 1,000 cm3/cm2/s or no greater than about 600 cm3/cm2/s.

Another aspect of the present disclosure provides an elastic article including the warp-knitted fabric according to one aspect of the present disclosure. In one aspect, the elastic article is selected from a group consisting of a sanitary product (such as a diaper), a medical product (such as a bandage), a sports product, underwear, and bedding.

Because the warp-knitted fabric of the present disclosure has in particular elasticity that is large in the warp direction (MD) and small in the weft direction (CD) and favorable breathability, it is particularly useful in a usage of, for example, a waist portion or a crotch portion of a diaper or a bandage.

### EXAMPLES

The illustrated aspects of the present invention are described further by giving examples below, but the present invention is not limited to these aspects.

### Evaluation Method

### 1.50% Tensile Stress, 100% Tensile Stress

Using tensilon, tensile stresses of when the warp-knitted fabric is elongated at an elongation rate of 50%, elongated at an elongation rate of 100%, and returned to an elongation rate of 50% after being elongated at an elongation rate of 100% are measured respectively.

### 2. Air Permeability

Air permeability is measured using a breathability tester (made by Toyo Seki Seisaku-Sho).

### Production of Warp-Knitted Fabric

Warp-knitted fabrics are produced according to the conditions indicated in table 1 and below.

Knitting machine: triple-guide-bar single-tricot knitting machine of 28 gauges (made by Karl Mayer)

Course count: 30

Yarn type: as indicated in table 1

Note that details of the yarn type are as below.

PU44T (polyurethane elastic yarn, 44 decitex)

56T24 (PET nonelastic yarn, 56 decitex)

84T36 and 84T36WL (PET nonelastic yarn, 84 decitex)

TABLE 1

| | | | | | | | 100% elongation → | |
|---|---|---|---|---|---|---|---|---|
| | | Knit | | | 50% elongation | 100% elongation | 50% elongation | Air permeability |
| | | structure | Yarn arrangement | Yarn type | (N/25 mm) | (N/25 mm) | (N/25 mm) | (cm3/cm2/s) |
| Example 1 | First yarn | 1-0/0-1 | 1 in, 8 out | PU44T | 0.31 | 0.68 | 0.15 | >400 |
| | Second yarn | 3-3/0-0 | 1 in, 2 out | PU44T | | | | |
| | Third yarn | 2-3/1-0 | Complete threading | 56T24 | | | | |
| Example 2 | First yarn | 1-0/0-1 | 1 in, 8 out | PU44T | 0.39 | 0.79 | 0.16 | >400 |
| | Second yarn | 3-3/0-0 | 1 in, 8 out | PU44T | | | | |
| | Third yarn | 2-3/1-0 | Complete threading | 84T36 | | | | |
| Example 3 | First yarn | 0-1/1-0 | 1 in, 8 out | PU44T | 0.45 | 1.21 | 0.16 | >400 |
| | Second yarn | 3-3/0-0 | 1 in, 2 out | PU44T | | | | |
| | Third yarn | 2-3/1-0 | Complete threading | 84T36 | | | | |
| Example 4 | First yarn | 0-1/1-0 | 1 in, 8 out | PU44T | 0.46 | 1.03 | 0.16 | >400 |
| | Second yarn | 3-3/0-0 | 1 in, 1 out | PU44T | | | | |
| | Third yarn | 2-3/1-0 | Complete threading | 84T36 | | | | |
| Example 5 | First yarn | 1-0/0-1 | 1 in, 8 out | PU44T | 0.42 | 0.87 | 0.14 | >400 |
| | Second yarn | 3-3/0-0 | 1 in, 1 out | PU44T | | | | |
| | Third yarn | 2-3/1-0 | Complete threading | 84T36 | | | | |
| Example 6 | First yarn | 1-0/0-1 | 1 in, 8 out | PU44T | 0.85 | 1.85 | 0.43 | >400 |
| | Second yarn | 3-3/0-0 | Complete threading | PU44T | | | | |
| | Third yarn | 2-3/1-0 | Complete threading | 84T36WL | | | | |
| Example 7 | First yarn | 1-0/0-1 | 1 in, 1 out | PU44T | 1.56 | 3.43 | 0.72 | >400 |
| | Second yarn | 3-3/0-0 | Complete threading | PU44T | | | | |
| | Third yarn | 2-3/1-0 | Complete threading | 84T36WL | | | | |

TABLE 2

| | | | | | | | 100% elongation → | Air |
|---|---|---|---|---|---|---|---|---|
| | | Knit | | | 50% elongation | 100% elongation | 50% elongation | permeability |
| | | structure | Yarn arrangement | Yarn type | (N/25 mm) | (N/25 mm) | (N/25 mm) | (cm³/cm²/s) |
| Comparative | First yarn | 1-2/1-0 | Complete threading | PU44T | 0.93 | 4.1 | 0.36 | >400 |
| example 1 | Second yarn | None | — | — | | | | |
| | Third yarn | 1-0/2-3 | Complete threading | 56T24 | | | | |
| Comparative | First yarn | 3-3/0-0 | Complete threading | PU44T | 0.71 | 1.73 | 0.37 | >400 |
| example 2 | Second yarn | None | — | — | | | | |
| | Third yarn | 2-3/1-0 | Complete threading | 84T36WL | | | | |

| | | | | | |
|---|---|---|---|---|---|
| Comparative | Acquired from Attento Sports Pants (trade name) made by | — | 2.7 | 0.7 | 30 |
| example 3 | Daio Paper Co. | | | | |
| Comparative | Acquired from Depend (registered trademark) Real Fit | — | 2.8 | 0.72 | 40 |
| example 4 | (registered trademark) made by Kimberly-Clark Co. | | | | |
| Comparative | Acquired from Lifree Slim Wear (trade name) made by | — | 9 | 0.3 | 300 |
| example 5 | Unicharm Co. | | | | |

Each warp-knitted fabric according to the examples has favorable elasticity and breathability and has no curling. Meanwhile, there is curling in comparative examples 1 and 2 (both being a double-guide-bar warp-knitted fabric), breathability is extremely low in comparative examples 3 and 4 (stacked bodies of a nonwoven fabric and a film), and in comparative example 5 (nonwoven fabric), while a tensile stress at a time of 100% elongation is extremely large, a tensile stress at a time of returning to an elongation rate of 50% after elongating at an elongation rate of 100% is extremely small and an elastic performance is degraded.

The warp-knitted fabric of the present disclosure can be applied favorably as an elastic article with a usage as a sanitary product, a medical product, a sports product, underwear, bedding, or the like.

### REFERENCE SIGNS LIST

1A, 1B, 1C First yarn
2A, 2B, 2C, 2D, 2E Second yarn
3A, 3B, 3C, 3D, 3E Third yarn

1. A warp-knitted fabric configured by a first yarn, a second yarn, and a third yarn, wherein
the first yarn and the second yarn are elastic yarns,
the third yarn is a nonelastic yarn, and
an amplitude A of the first yarn, an amplitude B of the second yarn, and an amplitude C of the third yarn satisfy relationships of A≤B and A≤C.

2. The warp-knitted fabric according to claim 1, wherein the first yarn is a back yarn, the second yarn is a middle yarn, and the third yarn is a front yarn.

3. The warp-knitted fabric according to claim 1, wherein the amplitude A of the first yarn is no greater than 2.

4. The warp-knitted fabric according to claim 1, wherein the amplitude B of the second yarn and the amplitude C of the third yarn are no greater than 5.

5. The warp-knitted fabric according to claim 1, wherein the first yarn is configured in a knit structure of a chain stitch.

6. The warp-knitted fabric according to claim 1, wherein at least a portion of the first yarn is configured according to a knit structure of 0-1/1-0.

7. The warp-knitted fabric according to claim 6, wherein the first yarn is configured according to the knit structure of 0-1/1-0.

8. The warp-knitted fabric according to claim 1, wherein the second yarn is configured according to a knit structure of 0-0/N-N, 0-1/N-N, N-N/1-0, or N-N/0-0; the third yarn is configured according to a knit structure of 1-0/(M−1)-M; and
1≤N≤5 and 1≤M≤5.

9. The warp-knitted fabric according to claim 8, wherein N and M are equal.

10. The warp-knitted fabric according to claim 1, wherein the first yarn and the second yarn each include no less than one type selected from a group consisting of a polyurethane and a polyether ester.

11. The warp-knitted fabric according to claim 1, wherein the third yarn includes no less than one type selected from a group consisting of a polyester, a polyolefin, a polyamide, an acrylic, cellulose, wool, and linen.

12. The warp-knitted fabric according to claim 1, wherein in a warp direction, a tensile stress at a time of elongation at an elongation rate of 100% is 0.4 N/25 mm to 6 N/25 mm.

13. The warp-knitted fabric according to claim 1, wherein in the warp direction, a tensile stress at a time of returning to an elongation rate of 50% after elongation at an elongation rate of 100% is 0.1 N/25 mm to 4 N/25 mm.

14. The warp-knitted fabric according to claim 1, wherein an air permeability is 10 cm3/cm2/s to 1,000 cm3/cm3/s.

15. An elastic article, comprising: the warp-knitted fabric according to claim 1; wherein the elastic article is selected from a group consisting of a sanitary product, a medical product, a sports product, underwear, and bedding.
